(19) United States
(12) Patent Application Publication
(10) Pub. No.: US 2016/0073915 A1
(43) Pub. Date: Mar. 17, 2016

(54) Wireless EKG System

(71) Applicant: Louis Felice, Selden, NY (US)

(72) Inventor: Louis Felice, Selden, NY (US)

(21) Appl. No.: 14/855,832

(22) Filed: Sep. 16, 2015

Related U.S. Application Data
(60) Provisional application No. 62/051,326, filed on Sep. 17, 2014.

(51) Int. Cl. A61B 5/0408; A61B 5/044; A61B 19/00; A61B 5/00

(52) U.S. Cl. CPC A61B 5/0408; A61B 5/0006; A61B 5/044; A61B 19/44; A61B 2019/442

(57) ABSTRACT

A wireless electrocardiogram (EKG) system having a plurality of wireless electrodes configured to wirelessly transmit EKG data to a display that is configured to display EKG data through a graphical EKG plot. Each wireless electrode includes a barcode thereon configured to allow a user to associate a set of wireless electrodes with a particular display. The display includes a barcode scanner, which is adapted to read and decode the barcode on each electrode to associate the electrode with the display. Each wireless electrode includes a housing configured to support an EKG sensor, a microprocessor adapted to process EKG signals from the EKG sensor for wireless transmission, and a wireless transmitter configured to transmit and encode EKG signals. The display includes a receiver configured to receive EKG data from the wireless transmitter and a microprocessor configured to generate the graphical EKG plot on the display.

### CROSS REFERENCE TO RELATED APPLICATION

This application claims the benefit of U.S. Provisional Application No. 62/051,326 filed on Sep. 17, 2014. The above identified patent application is herein incorporated by reference in its entirety to provide continuity of disclosure.

### FIELD OF THE INVENTION

The present invention relates to EKG systems. More specifically, the present invention provides a wireless EKG system having a plurality of wireless electrodes each having a barcode configured to allow a user to quickly associate a particular electrode or set of electrodes with a corresponding display.

### BACKGROUND OF THE INVENTION

Many health evaluations are conducted through the use of an electrocardiogram (hereinafter “EKG”). An EKG is typically used for evaluating a patient's cardiac activity, such as the patient's heart rate and heart rhythm. The EKG is also indicative of the conduction of low level electrical impulses across the myocardium. These low level impulses are also referred to as cardiac impulses. Conventionally, an EKG includes a plurality of sensors that are removably secured to the body of the patient. Typically, these sensors are removably adhered to the torso and chest regions of a patient. Conventional systems include three to twelve sensors.

The EKG measures low level voltage signals appearing between pairs of electrodes and processes these signals to provide a graphical plot of the detected signal voltage over a period of time. In this way, the EKG can assist physicians in diagnosing abnormalities of the heart. These abnormalities include abnormal heart rates or rhythms, abnormal size of parts of the heart, such as enlargement or atrophy, abnormal conduction pathways, and location of damaged muscle. However, a limitation of the conventional EKG equipment is due to the use of wired electrodes that are physically connected to a display, thereby limiting the mobility of the patient. In addition, another limitation of the conventional EKG equipment fails to allow a user to quickly identify and associate a particular set of electrodes with a specific display. Therefore, there exists a need for a wireless EKG system having a barcode on each wireless electrode and a barcode scanner configured to allow a user to associate a particular set of electrodes with a specific display.

Devices have been disclosed in the prior art that relate to wireless EKG systems. These include devices that have been patented and published in patent application publications. These devices include wireless EKG systems that can transmit information to a monitor or mobile device. These devices, however, fail to provide a system having a plurality of wireless electrodes each having a wireless transmitter and a barcode that allows a user to quickly associate a specific set of wireless electrodes with a particular display.

The present invention provides a wireless EKG system having a plurality of wireless electrodes configured to wirelessly communicate EKG data to a display that is configured to display EKG data through a graphical EKG plot. Each wireless electrode includes a barcode disposed thereon configured to allow a user to quickly associate a set of wireless electrodes with a particular display. The display includes a barcode scanner, which is adapted to read and decode the barcode on each electrode to associate an electrode with a display. Each wireless electrode includes a housing configured to support an EKG sensor, a microprocessor adapted to process EKG signals from the EKG sensor for wireless transmission, and a wireless transmitter configured to transmit and encode EKG signals. The display includes a receiver configured to receive EKG data from the wireless transmitter and a microprocessor configured to generate the graphical EKG plot on the display.

It is therefore submitted that the present invention is substantially divergent in design elements from the prior art, and consequently it is clear that there is a need in the art for an improvement to EKG systems. In this regard, the instant invention substantially fulfills these needs.

### SUMMARY OF THE INVENTION

In view of the foregoing disadvantages inherent in the known types of EKG systems now present in the prior art, the present invention provides a wireless EKG system wherein the same includes a barcode scanner and a barcode on each electrode of the system to allow a user to quickly associate a particular set of electrodes with a specific display.

It is therefore an object of the invention to provide a new and improved wireless EKG system that has all of the advantages of the prior art and none of the disadvantages.

Another object of the present invention is to provide a new and improved wireless EKG system having a plurality of electrodes configured to wirelessly communicate EKG data to a display that is configured to display the EKG data via a graphical EKG plot.

Yet another object of the present invention is to provide a new and improved wireless EKG system having a barcode disposed on each of the plurality of electrodes configured to associate the plurality of electrodes with a specific display.

Still yet another object of the present invention is to provide a new and improved wireless EKG system having a barcode scanner disposed on the display configured to read and decode the barcode on each electrode to identify a set of electrodes that is associated with the display.

A further object of the present invention is to provide a new and improved wireless EKG system having an EKG sensor, a microprocessor adapted to process EKG signals from the EKG sensor for wireless transmission, and a wireless transmitter configured to transmit and encode EKG signals.

Yet a further object of the present invention is to provide a new and improved wireless EKG system, wherein the display includes a receiver configured to receive EKG data from the wireless transmitter.

Still yet another object of the present invention is to provide a new and improved wireless EKG system, wherein the device may be readily fabricated from materials that permit relative economy and are commensurate with durability.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a front view of a wireless electrode.

FIG. 1B shows a rear view of a wireless electrode.

FIG. 2 shows a perspective view of the wireless EKG system.

FIG. 3 shows a schematic diagram of the wireless EKG system.

### DETAILED DESCRIPTION OF THE INVENTION

Reference is made herein to the attached drawings. Like reference numerals are used throughout the drawings to depict like or similar elements of the wireless EKG system. For the purposes of presenting a brief and clear description of the present invention, the preferred embodiment will be discussed as used for providing a wireless EKG system having a barcode scanner configured to scan a barcode on each electrode of the wireless EKG system to allow a user to associate a set of electrodes with a specific display so that the data collected by the wireless electrodes can be shown on the display. The figures are intended for representative purposes only and should not be considered to be limiting in any respect.

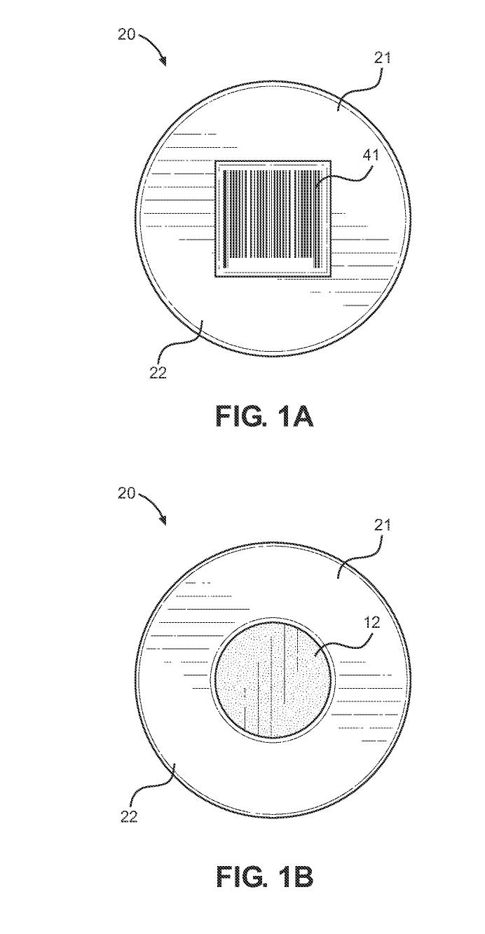

Referring now to FIGS. 1A and 1B, there are shown front and rear views of a wireless electrode of the EKG system. Each electrode 21 is enclosed in a housing 22 having a fastener 12 on a rear surface 14 thereof, wherein the fastener is adapted to allow the housing 22 to be removably secured to a desired portion of a user's body. In a preferred embodiment, the fastener 12 is an adhesive patch, however in alternate embodiments other fasteners 12 may be used. The housing 22 supports an EKG sensor therein, such as a direct contact voltage sensor, or a capacitive sensor, however, other alternative sensors are contemplated. In addition, the housing 22 supports a power source, a microprocessor adapted to process EKG signals from the EKG sensor for wireless transmission, and a wireless transmitter configured to transmit and encode EKG sensor signals to an EKG telemetry data receiver. In some embodiments, the housing 22 also supports an amplifier that is configured to provide an amplified EKG signal to a voltage to frequency converter. It is also contemplated that wireless communication can be achieved by a variety of means including, but not limited to, Bluetooth, WiFi, wireless carrier network, among others. These means for wireless communication are thus incorporated within the present disclosure.

Furthermore, each electrode 21 includes a barcode 41 on a front surface 13 thereof configured to encode a serial number. Each electrode 21 includes a unique barcode 41 that allows the electrodes 21 to be differentiated from one another and easily identified. The barcode 41 is adapted to be scanned by a scanner on a display, wherein the electrode 21 and display can wirelessly communicate once the barcode 41 has bene scanned. It is not desired at this time to limit the disclosed barcode 41 to a specific type of barcode. Rather, it is desired to disclose and claim a functional barcode that achieves the advantages and benefits described herein. It is anticipated that those of ordinary skill in the art will readily envision a variety of barcodes, such as one-dimensional barcodes and two-dimensional barcodes, which obtain the results described in the present disclosure. These variations do not alter the spirit of the inventive embodiments described and are deemed within the scope of the present disclosure.

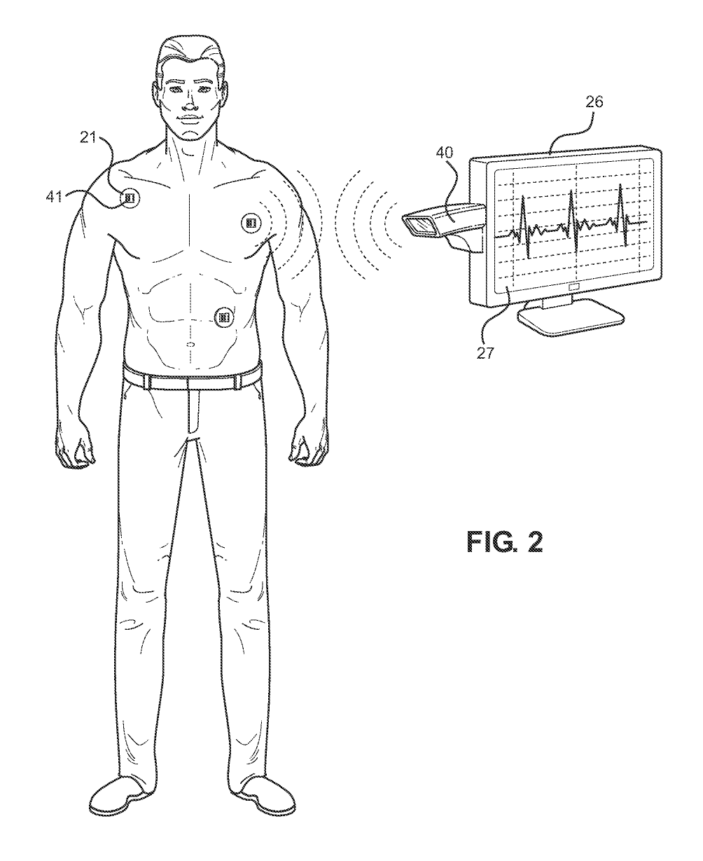

Referring now to FIG. 2, there is shown a perspective view of the wireless EKG system. In operation, one or more wireless electrodes 21 are removably secured to various portions of the patient's body, specifically the chest and torso of the patient. The scanner 40 can be used to scan the barcode 41 on each electrode 21 placed on the user's body. Once the barcode 41 of a specific electrode 21 has been scanned by the scanner 40, the display 26 and that electrode 21 can communicate wirelessly via the wireless transmitter on the electrode 21 and the wireless receiver on the display 26. The barcode 41 of multiple electrodes 21 can be scanned and the display 26 can then communicate with a plurality of electrodes 21, wherein the display 26 is able to differentiate between the various electrodes due to the unique barcodes provided on each electrode. The EKG data received at the receiver is processed and correlated with other EKG data received from the other wireless electrodes 21 via the microprocessor. The microprocessor is also configured to generate a graphical EKG plot 27 on the display 26 to display the results for analysis and examination by a health care professional.

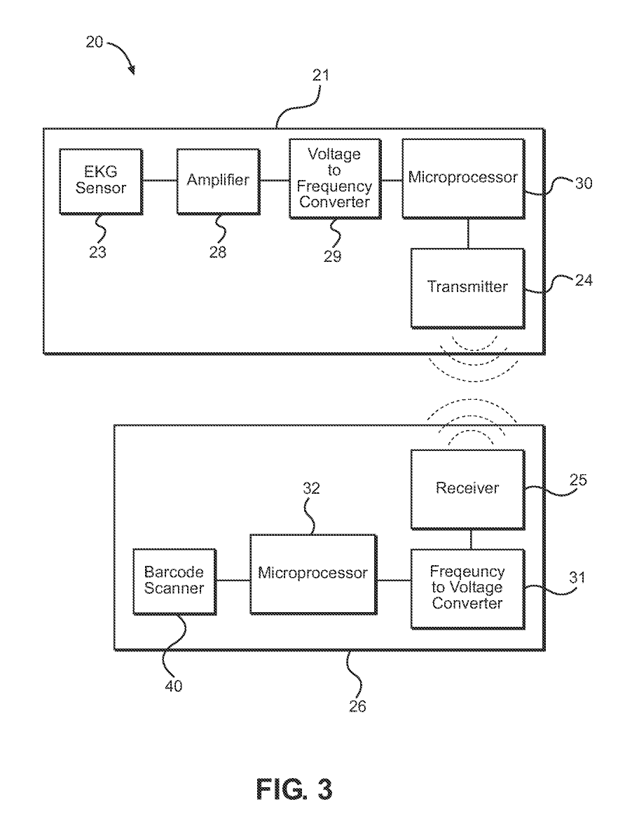

Referring now to FIG. 3, there is shown a schematic diagram of the wireless EKG system. The illustrated schematic diagram is intended to schematically illustrate one possible embodiment and is intended to be non-limiting. Other operating modes may be altered, enhanced, or modified without deviating from the spirit and scope of the present inventive disclosure. These other operating modes are deemed within the scope of the present disclosure. In the exemplary embodiment, the wireless electrode 21 includes the EKG sensor 23, the amplifier 28, the voltage to frequency converter 29, the microprocessor 30, and the wireless transmitter 24. The EKG sensor 23 is configured to transmit a low level EKG signal to the amplifier 28. The amplifier 28 is configured to provide an amplified EKG signal to the voltage to frequency converter 29. The voltage to frequency converter 29 is configured to convert the amplified EKG signal to a frequency encoded EKG signal, wherein the microprocessor 30 is configured to forward the frequency encoded EKG signal to the transmitter 24.

The display 26 includes the EKG telemetry data receiver 25 that is configured to receive the frequency encoded EKG signal from the transmitter 24. In addition, the display 26 also includes a frequency to voltage converter 31 that is configured to decode the received frequency encoded EKG signal into voltage signals, which are then processed by the display's microprocessor 32 to drive and generate a graphical EKG plot on the display 26.

Furthermore, the display 26 includes a barcode scanner 40 that is adapted to read and transmit the barcode on each electrode 21 to the microprocessor 32. The microprocessor is configured to decode and analyze the barcode so that a user can easily associate a plurality of electrodes 21 with a specific display 26. It is not desired for the present disclosure to limit the barcode scanner 40 disclosed to a specific type of barcode scanner. Rather, it is desired to claim and disclose a functional barcode scanner for obtaining the results and advantages described herein. Those of ordinary skill in the art will readily discern with the present disclosure before them that a variety of barcode scanners, such as pen-type readers and laser scanners, can be interchanged to achieve the advantages described herein without altering the scope of the invention. These variations and alterations are deemed to be within the spirit and scope of the inventive embodiments described herein.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A wireless EKG system, comprising:
a plurality of electrodes configured to wirelessly communicate EKG data to a display;
a barcode disposed on each of said plurality of electrodes;
a barcode scanner disposed on said display adapted to read and decode said barcode.

2) The wireless EKG system of claim 1, wherein each of said plurality of wireless electrodes includes a housing configured to support:
an EKG sensor;
a microprocessor adapted to process EKG signals from said EKG sensor for wireless transmission;
a wireless transmitter configured to transmit and encode said EKG signals.

3) The wireless EKG system of claim 2, wherein said housing further comprises:
an amplifier that is configured to provide an amplified EKG signal to a voltage to frequency converter that is configured to convert said amplified EKG signal to a frequency encoded EKG signal.

4) The wireless EKG system of claim 2, wherein said housing further comprises a power source.

5) The wireless EKG system of claim 1, wherein said display comprises a receiver configured to receive EKG data from said wireless transmitter.

6) The wireless EKG system of claim 1, wherein said display further comprises:
a frequency to voltage converter that is configured to decode said frequency encoded EKG signal to a voltage signal;
a microprocessor configured to process said voltage signal to drive and generate said graphical EKG plot on said display.
